(19) United States
(12) Patent Application Publication
(10) Pub. No.: US 2008/0031772 A1
(43) Pub. Date: Feb. 7, 2008

(54) METHOD OF INCREASING THE EFFECTIVENESS OF A CONTACT LENS CLEANING SOLUTION AGAINST RESISTANT STRAIN FUNGI

(76) Inventors: David J. Heiler, Avon, NY (US); Pramod Kumar Gupta, Pittsford, NY (US); Susan E. Burke, Batavia, NY (US); Suzanne F. Groemminger, Rochester, NY (US)

Correspondence Address: Bausch & Lomb Incorporated, One Bausch & Lomb Place, Rochester, NY, 14604-2701, US

(21) Appl. No.: 11/770,110

(22) Filed: Jun. 28, 2007

Related U.S. Application Data
(60) Provisional application No. 60/835,005, filed on Aug. 2, 2006.

(51) Int. Cl. A61L 12/14; C11D 3/48

(52) U.S. Cl. 422/28; 510/112

(57) ABSTRACT

A method of making a disinfecting solution that is effective at disinfecting contact lenses. The method includes the step of combining water, one or more antimicrobial agents and a reduced amount of solid content into a dissolved liquid mixture. The process results in an improved disinfection efficacy compared to a reference solution.

This application claims the benefit of Provisional Patent Application No. 60/835,005 filed Aug. 2, 2006 and is incorporated herein by reference.

### FIELD OF THE INVENTION

This invention relates to the field of cleaning and disinfecting contact lenses.

### DISCUSSION OF THE RELATED ART

Multipurpose cleaning solutions that function to clean, disinfect and condition contact lenses, were introduced in mid 1980's and rapidly became the choice of contact lens wearers for treatment of their lenses because of the combined effectiveness and convenience. Multipurpose cleaning solutions must effectively disinfect contact lenses to prevent ocular infection and at the same time effectively clean a contact lens. Nonionic surfactants have successfully been used in contact lens cleaning solutions because, in part, they have been compatible with antimicrobial agents. Typically, most multipurpose solutions for soft contact lenses have included a non-ionic surfactant since they have been on the market.

U.S. Pat. No. 4,820,352 discloses the use of poloxamine-type surfactants. Specifically, the superior effect of Tetronics 1107 was illustrated in protein and lipid cleaning. Concentrations of solutions ranging from 0.1 wt. % to 10 wt. % was disclosed in specific examples.

U.S. Pat. No. 5,209,865 discloses the combination of a poloxamine-type surfactant having an HLB value less than 7 in combination with a poloxamer-type surfactant having an HLB value less than 7. Specifically Tetronics® 1302 and Pluronics® L121 were an effective combination.

One requirement for approval of a multipurpose cleaning solution is to pass the “Stand-Alone Procedure for Disinfecting Products” (Stand-Alone Test) and is based on the Disinfection Efficacy Testing for contact lens care products under the Premarket Notification (510(k)) Guidance Document for Contact Lens Care Products dated May 1, 1997, prepared by the U.S. Food and Drug Administration, Division of Ophthalmic Devices. The primary criteria for a given disinfection period, corresponding to a potential minimum recommended disinfection period, is that the number of bacteria recovered per mL must be reduced by a mean value of not less than 3.0 logs within the given disinfection period. The number of mold and yeast recovered per ml must be reduced by a mean value of not less than 1.0 log within the minimum recommended disinfection time with no increase at four times the minimum recommended disinfection time. The Stand-Alone Test requires a solution to perform against five challenge organisms. They are Pseudomonas aeruginosa (ATCC 9027), Staphylococcus aureus (ATCC 6538), Serratia marcescens (ATCC 13880), Candida albicans (ATCC 10231) and Fusarium solani (ATCC 36031). These organisms are selected because they were representative of the microorganisms that are found in nature.

The FDA testing is designed to reasonably verify that effective disinfection will occur with a multipurpose solution should the patients follow the directions for use. However, potential infection may still arise due to two general factors. First, in nature, there is always a risk of resistant strains of microorganisms will arise in nature that will resist the disinfectants efficacy. Second, a number of contact lens wearers will not comply with the directions for use of a product. User abuse of product includes multiple use of a disinfecting solution. Rather than discard used solution, a non-compliant user will reinsert their contact lens into used solution multiple times sometimes adding only enough fresh solution to replace the amount of old solution that has evaporated or otherwise have been removed from the contact lens case.

Currently, there is a need for improved multipurpose disinfecting and cleaning solutions that are effective against resistant strains of microbes, even under conditions of misuse. The present invention addresses these and other needs.

### SUMMARY OF THE INVENTION

The present invention in one embodiment is in a method of manufacturing a disinfecting solution for disinfecting a contact lens inoculated with resistant strain fusarium microbes by contacting the medical device with a disinfecting solution comprising water, an antimicrobial agent a surfactant, and optionally a wetting agent, viscosity enhancing agent, a pH adjusting agent and a tonicity adjusting agent, the improvement comprising lowering the surfactant concentration of the disinfecting solution compared to a reference solution. The contact lens disinfecting solution has enhanced efficacy against resistant strain fusarium microbes compared to solutions without a reduced amount of solid content.

Generally, the amount of surfactant is reduced by 25 percent of the amount of surfactant in the reference solution. Typically, the solid content is reduced by 50 percent, 90 percent, 95 percent or 98 percent of the amount of surfactant in the reference solution.

The solution has enhanced efficacy against fungus relative to the reference solution. Generally, the solution has enhanced efficacy against resistant strain fusarium microbes.

### BRIEF DESCRIPTION OF THE DRAWING

Other advantages and features will be apparent from the below detailed description of the invention, example(s) and drawing.

FIG. 1 illustrates the influence of total surfactant concentration on the antimicrobial efficacy of 4.5 ppm Alexidine.2HCl

### DETAILED DESCRIPTION OF THE INVENTION

In one embodiment, there is a method of making a disinfecting solution that is effective at disinfecting contact lenses. The solution comprises water, an antimicrobial agent and one or more surfactants. The method comprises the step of combining the water, antimicrobial agent and a reduced amount of solid content into a dissolved liquid mixture. The disinfecting solution has enhanced efficacy against resistant strain fusarium microbes compared to solutions without a reduced amount of solid content. The process results in an improved disinfection efficacy compared to a reference solution.

Definitions

“Disinfecting solution,” as used herein, means an aqueous solution that is used for disinfecting a medical device.

“Medical device,” as used herein, means a device that is placed in contact with living tissue as a means of treating a condition in the patient.

“Inoculate,” as used herein, means to contact an object with a surface that is contaminated or thought to be contaminated with a resistant strain fusarium microbe.

“Resistant strain,” as used herein, means a strain of a microbe that is more resistant to disinfection or sterilization by a particular disinfecting solution than a commonly known microbe that is used as a challenge organism to represent a particular species. For the avoidance of doubt, ATCC 36031 fusarium is the commonly known microbe used as a challenge organism in FDA tests. A resistant strain of fusarium would be any strain that is more resistant to disinfection or sterilization by a particular disinfecting solution than ATCC 36031

“Fusarium microbes” or “fusarium,” as used herein, means any microbes that belong to the Fusarium species of fungi.

“Antimicrobial agent,” as used herein, means an additive to a solution that disinfects or preserves the solution, or is the primary agent that in combination with another agent that disinfects or preserves the solution against the FDA challenge organisms for disinfection or preservation.

“Ophthalmic formulation,” as used herein, means any formulation that is intended for use in contact with the tissue of the eye or ocular region.

“Ophthalmically safe,” as used herein, means that a material, formulation or solution does not cause unacceptable amount of ocular irritation, toxicity or harm to the eye or ocular region of a patient. By unacceptable it is meant that the level of toxicity is such that the material, formulation or solution would not be approved for use in contact with the ocular region of a patient.

“Solids content,” as used in this application, refers to the amount of solid or semi-solid ingredients that are added to a solution and is expressed as a wt. % of the total amount of solution. The solid content of a solution can be measured by adding totaling the weight of each solid item.

“Surfactant,” as used herein, means a surface active agent that is added to a contact lens to clean a lens of debris and remove lipids and other hydrophobic contaminants on the surface of a medical device.

“Wetting agent,” as used herein, means an agent that is added to improve the ability of a contact lens to retain moisture on the surface of the contact lens.

“Viscosity enhancing agent,” as used herein, means an agent that is used to increase the viscosity of a solution.

A “pH adjusting agent,” as used herein refers to any additive whose primary purpose is to adjust or stabilize the pH of a solution. For the avoidance of doubt, buffers acids and bases are pH adjusting agents regardless of whether the buffer potentiates the antimicrobial efficacy of a contact lens cleaning solution.

“Tonicity adjusting agent,” as used herein refers to any additive whose primary purpose is to adjust the tonicity or osmolality of a solution to a desired level.

“Ophthalmic lens,” as used herein means any lens that is placed in contact with the tissue of the eye or the ocular region of a patient.

“Anionic surfactant,” as used herein, means a surfactant that has a negatively charged ionic substituent group.

“Nonionic surfactant,” as used herein, generally means a surfactant that does not have a positively charged ionic substituent group and as it relates to polymeric surfactants, is a surfactant that has two or fewer charged groups. Specifically, Tetronics is a nonionic surfactant.

“Dilute,” as used herein, means to add a solvent to a solution to proportionally reduce the concentration of all solid contents of a solution.

“Reduction” or “lowering,” a particular solid component means to reduce or lower the concentration of at least one solid component in a solution compared to a reference solution regardless of whether or not the other solid components are proportionally reduced. “Reduced amount” refers to an amount of a solid component in a solution compared to the amount of the same or similar amount of a solid component in a solution.”

“Reference solution” refers to a previously existing solution in an iterative product development effort. The reference solution may be an existing product on the market or may be a previous experimental solution against which new solutions are compared.

“Dissolved liquid mixture,” as used herein means a mixture of solids in a liquid where the solids are dissolved.

“Enhanced efficacy,” of a solution refers to the effectiveness of the solution to disinfect a medical device or preserve a solution compared to a reference solution.

Composition or Formulation

The method and compositions of the present invention include a reduced amount of surfactant compared to the reference solution selected against which the solution of the present invention is compared in side-by-side testing. Thus, the reference solution must be a solution selected for side by side testing of the product that is the subject of this invention. For example, if ReNu with MoistureLoc is selected as a reference solution, then a reduced volume would be a volume that is less than found in ReNu with MoistureLoc. If ReNu Multiplus is selected as a reference solution, then a reduced volume would be a volume that is less than the volume found in ReNu Multiplus. Typically, the purpose of the subject solution would be to result in a formulation that is improvement over the reference solution. In one embodiment, the reference solution could be any formulation disclosed in the prior art that is used in side-by-side testing.

As noted, the composition of the present invention includes at least one surfactant, typically an anionic surfactant or a nonionic surfactant. Preferably the surfactant has advantages in terms of cleaning efficacy and comfort.

Surfactants are present in the subject compositions in a total amount of from approximately 0.001 to approximately 5.0 percent by weight based on the total weight of the composition, but more preferably from about 0.001 to about 3.0 percent by weight. Suitable surfactants include for example but are not limited to polyethers based upon poly(ethylene oxide)-poly(propylene oxide)-poly(ethylene oxide), i.e., (PEO-PPO-PEO), or poly(propylene oxide)-poly(ethylene oxide)-poly(propylene oxide), i.e., (PPO-PEO-PPO), or a combination thereof. PEO-PPO-PEO and PPO-PEO-PPO are commercially available under the trade names Pluronics™, R-Pluronics™, Tetronics™ and R-Tetronics™ (BASF Wyandotte Corp., Wyandotte, Mich.) and are further described in U.S. Pat. No. 4,820,352 incorporated herein in its entirety by reference. Suitable surfactants for use in the present composition should be soluble in the lens care solution, not become turbid, and should be non-irritating to eye tissues.

Another useful class of surfactants are the hydroxyalkylphosphonates (HAP), such as those disclosed in U.S. Pat. No. 5,858,937 (Richards et al.), available under the trade name Dequest® (Montsanto Co., St. Louis, Mo.), and most preferably Dequest® 2016.

Optionally, it may be desirable to include one or more water-soluble viscosity agents in the disinfecting solution. Because of the demulcent effect of viscosity agents, the same have a tendency to enhance the lens wearer's comfort by means of a film on the lens surface cushioning impact against the eye. Suitable viscosity agents include for example but are not limited to cellulose polymers like hydroxyethyl or hydroxypropyl cellulose, carboxymethyl cellulose, povidone, polyvinyl alcohol and the like. Viscosity agents may be employed in amounts ranging from about 0.01 to about 4.0 weight percent or less.

Compositions of the present invention when in disinfecting solution likewise include one or more buffers, or a buffering system in addition to the aminoalcohol buffer, to adjust the final pH of the solution. Suitable buffers include for example but are not limited to phosphate buffers, borate buffers, tris(hydroxymethyl)aminomethane (Tris) buffers, bis(2-hydroxyethyl)imino-

tris(hydroxymethyl)methane (bis-Tris) buffers, sodium bicarbonate, and combinations thereof. A suitable buffering system for example may include at least one phosphate buffer and at least one borate buffer, which buffering system has a buffering capacity of 0.01 to 0.5 mM, preferably 0.03 to 0.45, of 0.01 N of HCl and 0.01 to 0.3, preferably 0.025 to 0.25, of 0.01 N of NaOH to change the pH one unit. Buffering capacity is measured by a solution of the buffers only.

The pH of lens care solutions of the present invention is preferably maintained within the range of 5.0 to 8.0, more preferably about 6.0 to 8.0, most preferably about 6.5 to 7.8.

Disinfecting solutions of the present invention likewise include one or more tonicity agents to approximate the osmotic pressure of normal lachrymal fluids, which is equivalent to a 0.9 percent solution of sodium chloride or 2.5 percent glycerin solution. Examples of suitable tonicity agents include but are not limited to sodium and potassium chloride, dextrose, mannose, glycerin, calcium and magnesium chloride. These agents are typically used individually in amounts ranging from about 0.01 to 2.5 percent w/v and, preferably, from about 0.2 to about 1.5 percent w/v. Preferably, the tonicity agent is employed in an amount to provide a final osmotic value of 200 to 450 mOsm/kg and more preferably between about 220 to about 350 mOsm/kg, and most preferably between about 220 to about 320 mOsm/kg.

Disinfecting solutions of the present invention may optionally include one or more sequestering agents capable of binding to metal ions, which in the case of ophthalmic solutions, might otherwise react with protein deposits and collect on contact lenses. Suitable sequestering agents include for example but are not limited to ethylenediaminetetraacetic acid (EDTA) and its salts. Sequestering agents are preferably used in amounts ranging from about 0.01 to about 0.2 weight percent.

The compositions of the present invention are described in still greater detail in the examples that follow.

Methods of Use

The disinfecting solution of the present invention is useful in a number of contact lens disinfecting regimens. Basically, the disinfecting solution is contacted with a solution. In one embodiment, the contact lens is disinfected in a solution for a period that is a minimum of about one hour, two hours, three hours, four hours and six hours and a maximum of one day, eighteen hours, twelve hours and eight hours. Optionally, a rinse step is required. Optionally, a patient may be required to rub the contact lens between a finger and thumb of a wearer. In one embodiment, the regimen requires an alternative step of shaking the contact lens storage case containing disinfecting solution and a contact lens as an alternative to rubbing a contact lens.

Optionally, a rapid disinfection step is employed where the contact lens is placed in a solution that enables disinfection in less than 60 minutes, 40 minutes, 30 minutes or 20 minutes.

### EXAMPLES

### Example 1

### Effect of Solution Concentration on Biocidal Efficacy

To determine the biocidal efficacy of concentrated ReNu with MoistureLoc and ReNu MultiPlus solutions against the organisms Fusarium solani and Staphylococcus aureus.

### Part 1: Solution Preparation

A sample of ReNu with MoistureLoc was obtained and designated ML-1. Concentrated ReNu with MoistureLoc solutions were prepared by the following procedure with two and four times the concentration of each ingredient in ReNu with MoistureLoc and were designated ML-2 and ML-4 respectively. In a suitable container, 80% of the total volume of purified water is added. Boric acid, sodium phosphate (monobasic), sodium phosphate (dibasic), sodium chloride, HAP (30%), and Tetronic 1107 are measured and added to the measured water. Next, measured amounts of Pluronic® F127 and Polymer JR® are added to the solution. Water is added to 90% of the total volume with purified water. Then, the solution is mixed in an ice-chilled water bath overnight. The solution is autoclaved at 121° C. for 30 minutes. The solution is cooled and Alexidine stock solution is added. Sterile-filtered purified water is added to bring the water content to 100% of the total volume. The pH and osmolality of the solutions are determined and recorded in Table 1 Below.

A sample of ReNu MultiPlus is obtained and labeled MP-1. Concentrated solutions of ReNu MultiPlus are obtained with two and four times the concentration by the process below. The sample containing two times the solid content of ReNu MultiPlus is designated MP-2. The sample containing four times the solid content of ReNu MultiPlus is designated MP-4. In a suitable container, 80% of the total volume of purified water is added to the mixture vessel. Measured amounts of sodium chloride, EDTA, boric acid, sodium borate, Tetronic 1107, and HAP (30%) is measured and dissolved in the water. Purified water is added to 90% of the total amount of water. The solution is autoclaved at 121° C. for 30 minutes. Next, the solution is cooled and PHMB stock solution is added. Additional sterile-filtered purified water is added to bring the water content to 100% of the total volume. The pH and osmolality of the solutions are determined and recorded in Table 1 below.

TABLE 1
pH and Osmolality

| Solution | pH | Osmolality, mOsm/kg |
|---|---|---|
| ML-2 | 6.84 | >500 |
| ML-4 | 6.48 | >500 |
| MP-2 | 7.03 | >500 |
| MP-4 | 6.45 | >500 |
| ML-1 | 6.8-7.2 | 280-300 |
| MP-1 | 7.0-7.4 | 274-320 |

### Part 2: Biocidal Testing

Testing of MP-1, MP-2, MP-4, ML-1, ML-2 and ML-4 was tested against Staphylococcus aureus according to the following procedure. Six 0.1 mL samples of Fusarium solani (ATCC 36031) suspension is adjusted spectrophotometrically in Dulbecco's phosphate buffered saline with Tween 80 (DPBST) to approximately 1.0×10⁷-1.0×10⁸ colony forming units/mL (CFU/mL). The six samples were added each to separate tubes containing 10 ml of MP-1, MP-2, MP-4, ML-1, ML-2 and ML-4. After one hour, a 1 mL aliquot of each sample was removed and added to 9 mL of neutralizing Dey-Engley Broth (DEB). Then, 0.1 mL and 1 mL aliquots of DEB containing each of the samples aliquot was plated into TSA (Trypticase soy agar) to yield −1 and −2 dilutions, respectively. This was done in duplicate.
After four hours, a 1 mL aliquot of each sample was removed and added to 9 mL of DEB. Then, 0.1 mL and 1 mL aliquots of the DEB containing the sample aliquot was plated into TSA to yield −1 and −2 dilutions, respectively. This was done in duplicate. Results were calculated and reported in Table 2.
Testing of MP-1, MP-2, MP-4, ML-1, ML-2 and ML-4 was tested against Staphylococcus aureus according to the following procedure. Six 0.1 mL samples of Staphylococcus aureus (ATCC 6538) suspension is adjusted spectrophotometrically in Dulbecco's phosphate buffered saline with Tween 80 (DPBST) to approximately 1.0×10⁷-1.0×10⁸ colony forming units/mL (CFU/mL). The six samples were added each to separate tubes containing 10 ml of MP-1, MP-2, MP-4, ML-1, ML-2 and ML-4. After one hour, a 1 mL aliquot of each sample was removed and added to 9 mL of neutralizing Dey-Engley Broth (DEB). Then, 0.1 mL and 1 mL aliquots of DEB containing each of the samples aliquot was plated into TSA (Trypticase soy agar) to yield −1 and −2 dilutions, respectively. This was done in duplicate.
After four hours, a 1 mL aliquot of each sample was removed and added to 9 mL of DEB. Then, 0.1 mL and 1 mL aliquots of the DEB containing the sample aliquot was plated into TSA to yield −1 and −2 dilutions, respectively. This was done in duplicate. Results were calculated and reported in Table 2.

TABLE 2
Biocidal Efficacy Expressed in Log Reduction

| Log Reduction Values | |
|---|---|
| Staphylococcus | Fusarium |
| aureus | solani |

| Solution | 1 hr | 4 hr | 1 hr | 4 hr |
|---|---|---|---|---|
| MoistureLoc (2x) | <1 | <1 | <1 | <1.51 |
| MoistureLoc (4x) | <1 | <1 | <1 | <1 |
| MultiPlus (2x) | >5.24 | >5.24 | 4.51 | 4.81 |
| MultiPlus (4x) | 3.94 | 5.24 | 3.61 | >4.81 |
| MoistureLoc, | 4.64 | >5.24 | >4.81 | >4.81 |
| lot # GL5085 | | | | |

### CONCLUSION

The biocidal efficacy is significantly reduced for the concentrated MoistureLoc (2× and 4×), but it remains satisfactory for the concentrated MultiPlus (2× and 4×).

### Example 2

### Effect of Surfactant Concentration on Biocidal Efficacy

Several solutions were prepared according to the formulations illustrated in Table 3 and were labeled Formulations A-E. Other than surfactant concentration and salt concentration to compensate for tonicity, the amounts of other solids remained consistent from sample to sample.

TABLE 3
Composition of Test Formulations for Example 2

| | Formulation (% w/w) | | | | |
|---|---|---|---|---|---|
| | A | B | C | D | E |
| Boric Acid | 0.85 | 0.85 | 0.85 | 0.85 | 0.85 |
| NaH₂PO₄ | 0.15 | 0.15 | 0.15 | 0.15 | 0.15 |
| Na₂HPO₄ | 0.31 | 0.31 | 0.31 | 0.31 | 0.31 |
| HAP (30%) | 0.1 | 0.1 | 0.1 | 0.1 | 0.1 |
| Polymer JR | 0.02 | 0.02 | 0.02 | 0.02 | 0.02 |
| NaCl | 0.105 | 0.060 | 0.053 | 0.037 | 0.034 |
| Pluronic F127 | 3.0 | 3.0 | 4.5 | 5.0 | 5.5 |
| Tetronic 1107 | 0 | 1.5 | 0 | 0 | 0 |
| Alexidine | 4.5 | 4.5 | 4.5 | 4.5 | 4.5 |
| pH | 7.10 | 7.08 | 7.10 | 7.12 | 7.09 |
| Osmolality | 260 | 258 | 261 | 262 | 263 |
| (mOsm/kg) | | | | | |

The antimicrobial efficacy of each of the solutions for the chemical disinfection of contact lenses was evaluated. Microbial challenge inoculums were prepared using Pseudomonas aeruginosa (ATCC 9027), Staphylococcus aureus (ATCC 6538), Serratia marcescens (ATCC 13880), Candida albicans (ATCC 10231) and Fusarium solani (ATCC 36031).

The test organisms were cultured on appropriate agar and the cultures were harvested using sterile Dulbecco's Phosphate Buffered Saline plus 0.05 percent weight/volume polysorbate 80 (DPBST) or a suitable diluent and transferred to a suitable vessel. Spore suspensions were filtered through sterile glass wool to remove hyphal fragments. Serratia marcescens, as appropriate, was filtered through a 1.2 mu filter to clarify the suspension.

After harvesting, the suspension was centrifuged at no more than 5000×g for a maximum of 30 minutes at 20 to 25 degrees Celsius. The supernatent was poured off and resuspended in DPBST or other suitable diluent. The suspension was centrifuged a second time, and resuspended in DPBST or other suitable diluent. All challenge bacterial and fungal cell suspensions were adjusted with DPBST or other suitable diluent to 1×10⁷ to 1×10⁸ cfu/mL. The appropriate cell concentration may be estimated by measuring the turbidity of the suspension, for example, using a spectrophotometer at a preselected wavelength, for example 490 nm. One tube was prepared containing a minimum of 10 mL of test solution per challenge organism. Each tube of the solution to be tested was inoculated with a suspension of the test organism sufficient to provide a final count of 1×10⁵ to 1×10⁶ cfu/mL, the volume of the inoculum not exceeding 1 percent of the sample volume.

Dispersion of the inoculum was ensured by vortexing the sample for at least 15 seconds. The inoculated product was stored at 10 to 25 degrees Celsius. Aliquots in the amount of 1.0 mL were taken of the inoculated product for determination of viable counts after certain time periods of disinfection. The time points for the bacteria were, for example, 1, 2, 3 and 4 hours when the proposed regimen soaking time was four hours. Yeast and mold were tested at an additional timepoint of 16 hours (4 times the regimen time).

The suspension was mixed well by vortexing vigorously for at least 5 seconds. The 1.0 mL aliquots removed at the specified time intervals were subjected to a suitable series of decimal dilutions in validated neutralizing media. The suspensions were mixed vigorously and incubated for a suitable period of time to allow for neutralization of the microbial agent. The viable count of organisms was determined in appropriate dilutions by preparation of triplicate plates of trypticase soy agar (TSA) for bacteria and Sabouraud dextrose agar (SDA) for mold and yeast. The bacterial recovery plates were incubated at 30 to 35 degrees Celsius for two to four days. The yeast recovery plates were incubated at 20 to 30 degrees Celsius for two to four days. The mold recovery plates were incubated at 20 to 25 degrees Celsius for three to seven days.

The average number of colony forming units was determined on countable plates. Countable plates refer to 30 to 300 cfu/plates for bacteria and yeast, and 8 to 80 cfu/plates for mold except when colonies are observed only for the 10⁰ or 10⁻¹ dilution plates. The microbial reduction was then calculated at the specified time points and recorded as set forth below in Table 2. In order to demonstrate the suitability of the medium used for growth of test organisms and to provide an estimation of the initial inoculum concentration, inoculum controls were made by dispersing an identical aliquot of the inoculum into a suitable diluent, for example DPBST, using the same volume of diluent used to suspend the organism listed above. Following inoculation in a validated neutralizing broth and incubation for an appropriate period of time, the inoculum control must be between 1.0×10⁵ to 1.0×10⁶ cfu/mL.

The solutions were evaluated based on the performance requirement referred to as the “Stand-Alone Procedure for Disinfecting Products” (Stand-Alone Test) and is based on the Disinfection Efficacy Testing for contact lens care products under the Premarket Notification (510(k)) Guidance Document for Contact Lens Care Products dated May 1, 1997, prepared by the U.S. Food and Drug Administration, Division of Ophthalmic Devices. This performance requirement does not contain a rub procedure. This performance requirement is comparable to current ISO standards for disinfection of contact lenses (revised 1995). The Stand-Alone Test challenges a disinfecting product with a standard inoculum of a representative range of microorganisms and establishes the extent of viability loss at predetermined time intervals comparable with those during which the product may be used.

The primary criteria for a given disinfection period, corresponding to a potential minimum recommended disinfection period, is that the number of bacteria recovered per mL must be reduced by a mean value of not less than 3.0 logs within the given disinfection period. The number of mold and yeast recovered per ml must be reduced by a mean value of not less than 1.0 log within the minimum recommended disinfection time with no increase at four times the minimum recommended disinfection time.

TABLE 4
Stand Alone Test Results for Example 2

| | Formulations | | | | |
|---|---|---|---|---|---|
| Test Organisms | A | B | C | D | E |
| S. aureus | | | | | |
| 1 hr | 4.7 | 2.2 | 3.4 | 2.1 | 1.5 |
| 4 hr | >4.8 | 2.9 | 4.0 | 3.7 | 2.2 |
| P. aeruginosa | | | | | |
| 1 hr | 4.7 | >4.7 | 4.7 | >4.7 | 4.4 |
| 4 hr | >4.7 | >4.7 | >4.7 | >4.7 | >4.7 |
| S. marcescens | | | | | |
| 1 hr | >4.9 | 3.5 | 3.8 | 3.8 | 3.0 |
| 4 hr | >4.9 | >4.9 | 4.8 | 4.6 | 3.8 |
| C. albicans | | | | | |
| 1 hr | 4.3 | 2.9 | 3.1 | 2.9 | 2.1 |
| 4 hr | >4.6 | 4.6 | 4.2 | 3.8 | 3.4 |
| F. solani | | | | | |
| 1 hr | >4.3 | 4.2 | 4.0 | 3.8 | 3.2 |
| 4 hr | >4.3 | >4.3 | >4.3 | >4.3 | 3.8 |

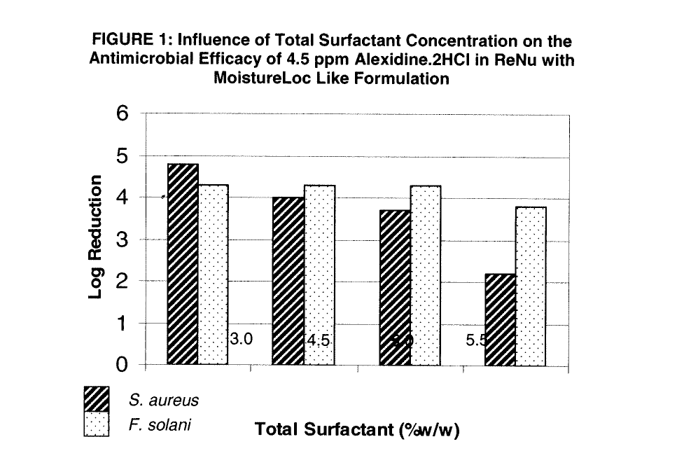

The results in FIG. 1, show a decrease in antimicrobial efficacy with increased concentrations of surfactant.

Although several specific embodiments have been depicted and described in detail, it will be apparent to those skilled in the relevant art that the specification, including the examples, are made without the intention of limiting the scope of the invention and that various modifications, additions, substitutions, and the like can be made without departing from the spirit of the invention are therefore considered to be within the scope of the invention as defined in the claims which follow.

What is claimed is:

1. In a method of manufacturing a disinfecting solution for disinfecting a contact lens inoculated with resistant strain fusarium microbes by contacting the contact lens with a disinfecting solution comprising water, an antimicrobial agent and a surfactant, a wetting agent, viscosity enhancing agent, a pH adjusting agent and a tonicity adjusting agent, the improvement comprising lowering the surfactant concentration of the disinfecting solution compared to a reference solution.

2. The method of claim 1, wherein the surfactant is a non-ionic surfactant.

3. The method of claim 1, wherein the amount of surfactant is reduced by 25 percent of the amount of surfactant in the reference solution.

4. The method of claim 1, further comprising increasing the tonicity of the solution.

5. The method of claim 1, wherein the solution has enhanced efficacy against resistant strain fusarium microbes.

6. The method of claim 1, further comprising adding a protein removal agent.

7. The method of claim 1, wherein the antimicrobial agent is a quaternary ammonium or biguamide antimicrobial agent.

8. The method of claim 1, wherein the surfactant is a poly(ethylene oxide)/poly(propylene oxide) containing surfactant

9. A method of making a disinfecting solution that is effective at disinfecting a contact lens comprising water, an antimicrobial agent and one or more surfactants, the method comprising the step of combining the water, antimicrobial agent and a reduced amount of solid content into a dissolved liquid mixture, wherein the disinfecting solution has enhanced efficacy against resistant strain fusarium microbes compared to solutions without a reduced amount of solid content.

10. The method of claim 9, wherein the surfactant is a non-ionic surfactant.

11. The method of claim 9, wherein the amount of surfactant is reduced by 25 percent of the amount of surfactant in the reference solution.

12. The method of claim 9, further comprising increasing the tonicity of the solution.

13. The method of claim 9, wherein the solution has enhanced efficacy against resistant strain fusarium microbes.

14. The method of claim 9, further comprising adding a protein removal agent.

15. The method of claim 9, wherein the antimicrobial agent is a quaternary ammonium or biguamide antimicrobial agent.

16. The method of claim 9, wherein the surfactant is a poly(ethylene oxide)/poly(propylene oxide) containing surfactant.

17. The method of claim 9, wherein the surfactant is a poloxamer surfactant.
